(19) United States
(12) Patent Application Publication
(10) Pub. No.: US 2020/0009027 A1
(43) Pub. Date: Jan. 9, 2020

(54) TEXTURED COMPOSITIONS

(71) Applicant: BASF SE, Ludwigshafen am Rhein (DE)

(72) Inventors: Heidi Riedel, Düsseldorf-Holthausen (DE); Robert Jung, Düsseldorf-Holthausen (DE); Helga Gondek, Düsseldorf-Holthausen (DE)

(21) Appl. No.: 16/468,023

(22) PCT Filed: Nov. 29, 2017
(86) PCT No.: PCT/EP2017/080814

§ 371 Date: Jun. 10, 2019

(30) Foreign Application Priority Data
Dec. 13, 2016 (EP) 16203639.6

(51) Int. Cl. A61K 8/06; A61K 8/60; A61K 8/19; A61K 8/36; A61K 8/34; A61K 8/92; A61K 8/37; A61K 8/31; A61K 8/81; A61Q 19/00; A61Q 17/04

(52) U.S. Cl. CPC A61K 8/062; A61K 8/60; A61K 8/19; A61K 8/361; A61K 8/342; A61K 8/922; A61Q 17/04; A61K 8/31; A61K 8/375; A61K 8/345; A61K 8/8147; A61K 8/347; A61Q 19/005; A61K 8/37

(57) ABSTRACT

The present invention describes compositions which form particular textures in oil-in-water systems and are used especially in the preparation of cosmetic products.

### 1. FIELD OF THE INVENTION

The present invention relates to textured compositions, especially for the cosmetics industry, which form particular molecular structures in an oil-in-water system. These compositions form interesting textures which can be classified as glassy-translucent to milky-gel-like in terms of appearance.

### 2. BACKGROUND OF THE INVENTION

A clear view of the requirements of the consumer and the identification of future trends are critical for the development of successful body care products. With customized products and solutions for cosmetics, the opportunities and possibilities lying within these new trends should be exhausted.

These new trends also include the development of textures of cosmetic products. Textures influence the wearing and application comfort of cosmetic products and impart an impression to the consumer with regard to the sensory properties of the product.

EP 1 212 042 B1 describes a structuring composition which forms a two-layer lamellar gel network in an oil-in-water system. This composition comprises a cationic swelling agent, which takes the form of a fatty acid adduct of amidopropyldimethyl-2-hydroxyethylammonium halide and a gelling agent mixture of emulsifiers having a low HLB value, which are selected from fatty alcohols and fatty esters. The ratio of the anionic emulsifier to the fatty alcohol is set exceptionally low. These product structuring compositions may be used in body care formulations for structure formation.

WO 98/44896 A1 discloses oil-in-water lamellar emulsions, the oil phase of which consists of oil mixtures having a high polarity.

Cosmetic oil-in-water emulsions are typically based on a microstructure of finely-divided oil droplets within an outer water phase. These emulsions are characterized by a homogeneous white texture. However, there is increasingly a need for novel experiences during application of care emulsions.

The object of the present invention consists in providing unusual textures which contrast optically from the homogeneous white oil-in-water emulsions and offer novel sensory experiences to the consumer when applying the product to the skin.

### 3. SUMMARY OF THE INVENTION

The present invention relates to a textured composition which forms a lamellar and/or crystalline gel network in an oil-in-water system and which has the constituents:
0.01 to 3.00 wt % of at least one anionic emulsifier, and
0.15 to 9.00 wt % of at least one consistency regulator,
0.01 to 5.00 wt % of at least one hydrocolloid, and
3.00 to 15.0 wt % of at least one oil and/or wax, and
made up to 100 wt % with water,
wherein the anionic emulsifier comprises at least one emulsifier from the group comprising salts of fatty acids, salts of stearoyl lactic acid, salts of stearoyl glutamic acid or alkyl glutamates, alkyl phosphates, alkyl sulfates, alkyl sarcosinates, salts of alkylsulfosuccinic acid and salts of citric acid esters, the ratio of anionic emulsifier to consistency regulator is in the range from 10:333 to 10:6 and
the oil or the oil mixtures have a medium polarity and an interfacial tension IFT of 25 to 45 mN/m.

The inventive compositions exhibit a broad range of textures extending from glassy-translucent to milky-gel-like textures. The respectively desired texture within this range can be adjusted through the selection of the emulsifier system (type of anionic emulsifier and type of consistency regulator) and also through the proportion and polarity of the oil/wax.

The invention also relates to a textured composition which forms a finely dispersed droplet distribution having lamellar and/or crystalline structures in an oil-in-water system and which has the constituents:
an emulsifier combination composed of
0.01 to 5.00 wt % of at least one anionic emulsifier, and
0.15 to 9.00 wt % of at least one consistency regulator,
0.01 to 5.00 wt % of at least one hydrocolloid, and
3.00 to 15.0 wt % of at least one oil and/or wax, and
made up to 100 wt % with water,
wherein the anionic emulsifier comprises at least one emulsifier from the group comprising salts of fatty acids, salts of stearoyl lactic acid, salts of stearoyl glutamic acid or alkyl glutamates, alkyl phosphates, alkyl sulfates, alkyl sarcosinates, salts of alkylsulfosuccinic acid and salts of citric acid esters, and the emulsifier combination is 2.00 to 8.00 wt % and the ratio of anionic emulsifier to consistency regulator is 1:1 to 1:2 and the oil or the oil mixtures have a medium polarity and an interfacial tension IFT of 25 to 45 mN/m.

This composition gives a smooth milky-cloudy oil-in-water emulsion.

All amounts relate to 100% (total amount) of the textured composition.

All textured compositions are characterized by an outstanding storage stability over a period of up to 3 years. Even at elevated temperatures (>40° C.) or variable temperatures (−5° C. to 40° C.) as well as freezing temperatures, a storage stability of up to 6 months is achieved.

The present invention also relates to a process for preparing these textured compositions and textured cosmetic compositions.

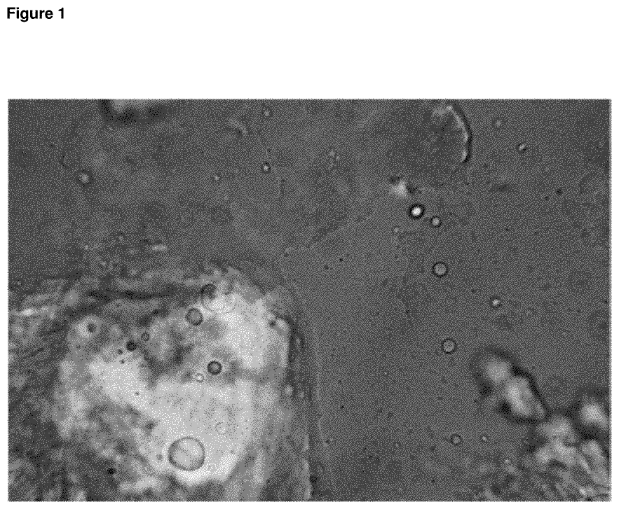

A lamellar and a crystalline, or a combined lamellar/crystalline, gel network structure are described in FIGS. 1 to 3.

The figures show:

FIG. 1 a micrograph of an inventive oil-in-water emulsion having crystalline gel network at a magnification of 400:1;

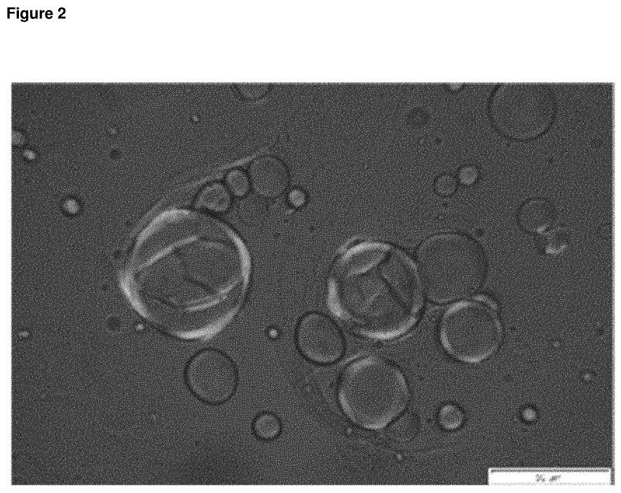

FIG. 2 a micrograph of an inventive oil-in-water emulsion having lamellar gel network at a magnification of 400:1;

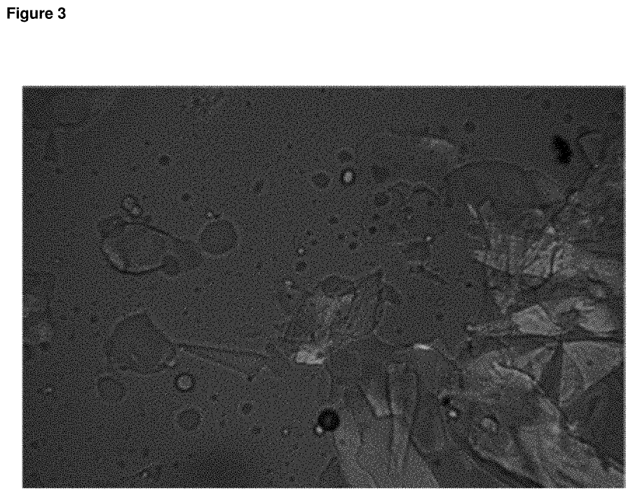

FIG. 3 a micrograph of an inventive oil-in-water emulsion having lamellar/crystalline gel network at a magnification of 400:1.

### DESCRIPTION OF THE INVENTION

The present invention describes compositions which form a lamellar and/or crystalline gel network in an oil-in-water system. The resulting texture has a translucent, glassy, visual effect. These compositions are stabilized by the lamellar gel network structure which contrasts with the classical emulsion structure having oil droplets finely distributed in the outer water phase. The compositions are not homogenized, but rather stirred under moderate conditions and are characterized by an exceptional long-term stability.

In a preferred embodiment of the present invention, the at least one consistency regulator is selected from the group of fatty alcohols, fatty acids (unsaponified), glyceryl mono-, di- and/or tri-fatty acid esters, sorbitan mono-, di-, tri- and/or poly-fatty acid esters, sucrose mono-, di-, tri- and/or poly-fatty acid esters, pentaerythrityl mono- and/or di-fatty acid esters.

It has proved advantageous if at least one consistency regulator is present at a concentration of 0.5 to 6.00 wt % in the composition.

Fatty alcohols for the consistency regulator are understood to mean primary aliphatic alcohols of the formula (I),

R—OH (I)

where R signifies an aliphatic, linear or branched alkyl radical having 6 to 22 carbon atoms, preferably 14 to 20 carbon atoms, having 0 and/or 1, 2 or 3 double bonds.

Typical examples for the fatty alcohol are caproic alcohol, caprylic alcohol, 2-ethylhexyl alcohol, capric alcohol, lauryl alcohol, isotridecyl alcohol, myristyl alcohol (Lanette® 14, Lanette® 14/MB), cetyl alcohol (Lanette® 16), palmoleyl alcohol, stearyl alcohol (Lanette® 18), cetearyl alcohol (Lanette® D), isostearyl alcohol, oleyl alcohol, elaidyl alcohol, petroselyl alcohol, linolyl alcohol, linolenyl alcohol, elaeostearyl alcohol, arachyl alcohol, gadoleyl alcohol, behenyl alcohol (Lanette® 22), erucyl alcohol and brassidyl alcohol and technical grade mixtures thereof, which are obtained, for example, in the high pressure hydrogenation of technical grade methyl esters based on fats and oils or aldehydes from Roelen's oxo synthesis and also as monomer fraction in the dimerization of unsaturated fatty alcohols. Preference is given to technical grade fatty alcohols having 12 to 18 carbon atoms such as, for example, coconut, palm, palm kernel or tallow fatty alcohols. Very particularly preferred fatty alcohols are cetyl alcohol, stearyl alcohol and cetearyl alcohol.

Further consistency regulators are glyceryl laurate, such as Monomuls 90-L-12, glyceryl oleate, such as Monomuls 90-O 18, glyceryl stearate, such as Cutina GMS V and Cutina GMS V/MB, glycol distearate, such as Cutina AGS, sorbitan stearate, such as Dehymuls SMS, pentaerythrityl distearate, such as Cutina PES, sucrose polystearate (and) cetyl palmitate, such as Emulgade Sucro Plus and sucrose polystearate (and) hydrogenated polyisobutene, such as Emulgade Sucro.

The hydrocolloid present in the textured composition is selected from the group comprising acrylic acid and acrylic acid derivatives, carbohydrates such as cellulose and natural rubber and derivatives thereof. Preferred hydrocolloids are polyacrylates (Cosmedia® SP and Cosmedia® ACE, BASF), carbomer (Rheocare® C Plus, BASF), acrylate copolymer (Rheocare® TTA). A particularly preferred hydrocolloid is a carbomer.

The concentration of the hydrocolloid in the composition is preferably in a range from 0.1 to 1.00 wt %.

According to the invention, the anionic emulsifier is at least one emulsifier from the group comprising salts of fatty acids, salts of stearoyl lactic acid, salts of stearoyl glutamic acid or alkyl glutamates, alkyl phosphates, alkyl sulfates, alkyl sarcosinates, salts of alkylsulfosuccinic acid and salts of citric acid esters. Preferred anionic emulsifiers are glyceryl stearate/stearic acid (Cutina® FS 45, BASF), sodium stearoyl glutamate (Eumulgin® SG, BASF), disodium cetearyl sulfosuccinate (Eumulgin® Prisma, BASF), sodium cetearyl sulfate (Lanette® E, Lanette® N, Lanette® SX BASF). Particularly preferred anionic emulsifiers are glyceryl stearate/stearic acid, sodium stearoyl glutamate and disodium cetearyl sulfosuccinate.

The oil and/or wax in the textured composition according to the invention is selected from the group comprising fatty acid esters, hydrocarbons, Guerbet alcohols, tri- or partial glycerides, mono-/dialkyl ethers, mono-/dialkyl carbonates, oil-soluble UV filters, fatty alcohol ethers, microcrystalline waxes, mineral oil, silicone oil, natural vegetable oils and mixtures thereof. The concentration of the oil in the composition is 3.00 to 15.00 wt %, preferably 5.00 to 15.00 wt %.

Preferred oils and/or waxes are Guerbet alcohols based on fatty alcohols having 6 to 18, preferably 8 to 10 carbon atoms (Eutanol G, Eutanol G 16), esters of linear C₆-C₂₂-fatty acids with linear or branched C₆-C₂₂-fatty alcohols or esters of branched C₆-C₁₃-carboxylic acids with linear or branched C₆-C₂₂-fatty alcohols such as, e.g. myristyl myristate (Cetiol MM), myristyl palmitate, myristyl stearate, myristyl isostearate, myristyl oleate, myristyl behenate, myristyl erucate, cetyl myristate, cetyl palmitate (Cutina CP), cetyl stearate, cetyl isostearate, cetyl oleate, cetyl behenate, cetyl erucate, stearyl myristate, stearyl palmitate, stearyl stearate, stearyl isostearate, stearyl oleate, stearyl behenate, stearyl erucate, isostearyl myristate, isostearyl palmitate, isostearyl stearate, isostearyl isostearate, isostearyl oleate, isostearyl behenate, isostearyl oleate, isopropyl myristate, isopropyl palmitate, oleyl myristate, oleyl palmitate, oleyl stearate, oleyl isostearate, oleyl oleate, oleyl behenate, oleyl erucate (Cetiol J 600), behenyl myristate, behenyl palmitate, behenyl stearate, behenyl isostearate, behenyl oleate, behenyl behenate, behenyl erucate, erucyl myristate, erucyl palmitate, erucyl stearate, erucyl isostearate, erucyl oleate, erucyl behenate and erucyl erucate, ethylhexyl stearate (Cetiol 868), hexyl laurate (Cetiol A), C12.15 alkyl benzoate (Cetiol AB), dibutyl adipate (Cetiol B), coco caprylate (Cetiol C5), coco caprylate/caprate (Cetiol LC, Cetiol C 5C), propylheptyl caprylate (Cetiol Sensoft), cetearyl isononanoate (Cetiol SN), decyl oleate (Cetiol V), cetearyl ethylhexanoate (Luvitol EHO). Also suitable are esters of C₁₈-C₃₈-alkylhydroxycarboxylic acids with linear or branched C₆-C₂₂-fatty alcohols, especially dioctyl malate, esters of linear and/or branched fatty acids with polyhydric alcohols (such as e.g. propylene glycol, dimerdiol or trimertriol) such as propylene glycol dicaprylate/dicaprate (Myritol PGDC) and/or Guerbet alcohols, triglycerides based on C₆-C₁₀-fatty acids, liquid mono-/di-/triglyceride mixtures based on C₆-C₁₈-fatty acids (Myritol 331, Myritol 312, Myritol 318), esters of C₆-C₂₂-fatty alcohols and/or Guerbet alcohols with aromatic carboxylic acids, especially benzoic acid, esters of C₂-C₁₂-dicarboxylic acids with linear or branched alcohols having 1 to 22 carbon atoms or polyols having 2 to 10 carbon atoms and 2 to 6 hydroxyl groups, vegetable oils, branched primary alcohols, substituted cyclohexanes, linear and branched C₆-C₂₂-fatty alcohol carbonates such as e.g. dicaprylyl carbonate (Cetiol® CC), Guerbet carbonates based on fatty alcohols having 6 to 18, preferably 8 to 10 carbon atoms, esters of benzoic acid with linear and/or branched C₆-C₂₂-alcohols (e.g. Finsolv® TN, Cetiol AB), linear or branched, symmetrical or asymmetrical dialkyl ethers having 6 to 22 carbon atoms per alkyl group, such as e.g. dicaprylyl ether (Cetiol® OE), ring-opening products of epoxidized fatty acid esters with polyols (Cetiol E). Further suitable emollients are vegetable oils (Cegesoft GPO, Cegesoft PFO, Cegesoft PS 6, Cegesoft SBE, Cegesoft SH) and mixtures thereof (Cegesoft VP), silicone oils, hydrocarbons such as Cetiol Ultimate, hydrogenated polyisobutene (Luvitol Light), mineral oils, isoparaffins, paraffins.

Particularly preferred oils are high or medium polarity oils such as, for example, coco caprylate/caprate (Cetiol® LC), ethylhexyl palmitate (CEGESOFT® C24), Elaeis guineensis palm oil (CEGESOFT® GPO), Passiflora incarnata seed oil (CEGESOFT® PFO), Olus oil (CEGESOFT® PS 6), ethylhexyl stearate (CETIOL® 868), hexyl laurate (CETIOL® A), C12-15 alkyl benzoate (CETIOL® AB), dibutyl adipate (CETIOL® B), coco caprylate (CETIOL® C5), coco caprylate/caprate (CETIOL® C 5C), dicaprylyl carbonate (CETIOL® CC), ethylhexyl cocoate (and) Cocos nucifera oil (CETIOL® COCO), PPG-15 stearyl ether (CETIOL® E), oleyl erucate (CETIOL® J 600), hexyldecanol (and) hexyldecyl laurate (CETIOL® PGL), caprylyl caprylate/caprate (CETIOL® RLF), propylheptyl caprylate (CETIOL® SENSOFT), cetearyl isononanoate (CETIOL® SN), decyl oleate (CETIOL® V), octyldodecanol (EUTANOL® G), hexyldecanol (EUTANOL® G16), hexyldecyl stearate (EUTANOL® G165), isopropyl myristate, isopropyl palmitate, cetearyl ethylhexanoate (LUVITOL® EHO), capryl/capric triglyceride (MYRITOL® 312), capryl/capric triglyceride (MYRITOL® 318), coco glyceride (MYRITOL® 331).

An especially preferred oil is coco caprylate/caprate.

The compositions contain oils or oil mixtures which have a medium polarity and an interfacial tension IFT of 25 to 45 mN/m.

The polarity or interfacial tension of an oil is measured as follows:

The polarity of an oil is defined as the interfacial tension thereof (IFT in mN/m) versus water and is measured using the pendant drop method at a temperature of 23 +/−2° C. A Dataphysics OCAH 200 high-speed contact angle measuring system having a 25 gauge cannula (DataPhysics Instruments GmbH, Filderstadt, Germany) is used for the measurement. The cannula is used to form a water droplet in a cuvette filled with the respective oil, and the droplet size is adjusted in 5 μl steps up to the maximum stable volume, in order to ensure the highest sensitivity for the method. For evaluating the droplet size, the LaPlace-Young method is used and the required densities are determined with an oscillating U-tube density measuring device. The measurement is repeated 10 times and the IFT is given as the mean of these 10 measurements with the standard deviation thereof.

Typical examples of fats are glycerides, i.e. solid vegetable or animal products which are composed essentially of mixed glycerol esters of higher fatty acids. Fatty acid partial glycerides, i.e. technical-grade mono- and/or diesters of glycerol with fatty acids having 12 to 18 carbon atoms, such as, for instance, glycerol mono/dilaurate, -palmitate or -stearate, are also possible for this purpose. Possible waxes are, inter alia, natural waxes, such as, for example, candelilla wax, carnauba wax, japan wax, esparto grass wax, cork wax, guaruma wax, rice bran oil wax, sugarcane wax, ouricury wax, montan wax, beeswax, shellac wax, spermaceti, lanolin (wool wax), uropygial grease, ceresin, ozokerite (earth wax), petrolatum, paraffin waxes, micro waxes; chemically modified waxes (hard waxes), such as, for example, montan ester waxes, Sasol waxes, hydrogenated jojoba waxes, and also synthetic waxes, such as, for example, polyalkylene waxes and polyethylene glycol waxes. As well as the fats, fat-like substances, such as lecithins and phospholipids, are also possible as additives. Examples of natural lecithins which may be mentioned are the cephalins, which are also referred to as phosphatidic acids and are derivatives of 1,2-diacyl-sn-glycerol-3-phosphoric acids. In contrast, phospholipids are usually understood to mean mono- and preferably diesters of phosphoric acid with glycerol (glycerol phosphates), which are generally counted among the fats. In addition, sphingosines or sphingolipids are also possible.

The emulsifier combination of anionic emulsifier and consistency regulator in the textured composition according to the invention is preferably 1.5 to 6.0 wt %.

It has proved to be according to the invention that the selection of the consistency regulator influences the gel network in the composition. When using fatty alcohols as consistency regulator, a lamellar gel network is formed, while the selection of an unsaponified fatty acid leads to the formation of a crystalline gel network. When using esters as consistency regulator, a mixed gel network structure is formed, i.e. a lamellar and crystalline gel network.

FIG. 1 shows a light micrograph (400:1) of a gel-like oil-in-water emulsion having a crystalline gel network structure. This composition is highly translucent (transparent) with a clear and/or pearly-marbled appearance.

A lamellar gel network structure can be seen using the light micrograph (400:1) of the oil-in-water emulsion of FIG. 2. Milky textures can be perceived, which can be described as light or creamily rich, with a sorbet-like character.

A mixed gel network structure can be seen in a light micrograph (400:1) of a further oil-in-water emulsion in FIG. 3. The texture of this oil-in-water emulsion shifts between translucent and milky-gel-like.

The inventive textured compositions have translucency (transparency). The transmittance of the textured compositions is 3.9 to 50%, preferably 3.9 to 20%, at 25° C.

The transmittance describes the proportion of the incident radiation flux or light flux that completely penetrates a transparent system.

The inventive textured compositions are prepared by simple stirring of the constituents without homogenization. In this manner, the lamellar or crystalline network structure can form. To this end, the constituents are combined after heating to 75 to 85° C. and are stirred after the phase combination with slow stirring at a speed of <600 rpm until room temperature is reached. The stirring speed during stirring is preferably 300 rpm. The resulting composition is storage-stable and maintains its texture for a period of up to 3 years.

Surprisingly, various further textures different therefrom may be prepared by modifying the amounts or concentration of the constituents present in the textured composition of the invention. For instance, a textured gel may be prepared by omitting the oil phase.

The textured compositions according to the invention are excellent for use in the field of cosmetics. The present invention therefore also relates to textured cosmetic compositions which are present in the textures described above. These cosmetic compositions further comprise cosmetically active constituents from the group comprising pigments, plant extracts, peptides, proteins, marine atelocollagen, phytoceramides, phytosterols, polyphenols, polyols, urea, hyaluronic acid, sugars and sugar derivatives, sodium PCA, vitamins, UV light protection filters, antioxidants, biogenic active ingredients, self-tanning agents, preservatives, complexing agents, perfume oils, vegetable oils, antiperspirants, esterase inhibitors, neutralizing agents, bactericides and mixtures thereof.

The compositions according to the invention can be in the form of creams, milk, lotions, gels, sticks, conditioners, sprays, serum, aerosol foam, pump foam, pastes or waxes.

The textured compositions are highly suitable for skincare for the face, body, hands, area around the eyes and décolletage, as sunscreen compositions and for skincare for babies and also for the care of unwell and sensitive skin.

Examples of cosmetically active constituents are described hereinafter.

### Antiperspirants

Antiperspirants are salts of aluminum, of zirconium or of zinc. Such suitable antihydrotic active ingredients are, e.g. aluminum chloride, aluminum chlorohydrate, aluminum dichlorohydrate, aluminum sesquichlorohydrate and complexes thereof, for example with 1,2-propylene glycol, aluminum hydroxyallantoinate, aluminum chloride tartrate, aluminum zirconium trichlorohydrate, aluminum zirconium tetrachlorohydrate, aluminum zirconium pentachlorohydrate and complexes thereof, for example with amino acids such as glycine.

Preference is given to using aluminum chlorohydrate, aluminum zirconium tetrachlorohydrate, aluminum zirconium pentachlorohydrate and complexes thereof.

### Esterase Inhibitors

In the presence of perspiration in the underarm region, bacteria form extracellular enzymes—esterases, preferably proteases and/or lipases—which cleave esters present in the perspiration and thus release odorants. Suitable esterase inhibitors are preferably trialkyl citrates such as trimethyl citrate, tripropyl citrate, triisopropyl citrate, tributyl citrate and especially triethyl citrate (Hydagen® CAT, BASF AG, Dusseldorf). The substances inhibit enzyme activity and hence reduce odor formation. Further substances which are possible esterase inhibitors are sterol sulfates or phosphates, for example sulfates or phosphates of lanosterol, of cholesterol, of campesterol, of stigmasterol and of sitosterol, dicarboxylic acids and esters thereof, for example glutaric acid, monoethyl glutarate, diethyl glutarate, adipic acid, monoethyl adipate, diethyl adipate, malonic acid and diethyl malonate, hydroxycarboxylic acids and esters thereof, for example citric acid, malic acid, tartaric acid or diethyl tartrate, and zinc glycinate.

### Bactericidal or Bacteriostatic Active Ingredients

Typical examples of suitable bactericidal or bacteriostatic active ingredients are especially chitosan and phenoxyethanol. 5-Chloro-2-(2,4-dichlorophenoxy)phenol has also been found to be particularly effective, and is sold under the Irgasan® brand by Ciba-Geigy, Basle, Switzerland. Suitable germicides are in principle all substances which act effectively against Gram-positive bacteria, e.g. 4-hydroxybenzoic acid and salts and esters thereof, N-(4-chlorophenyl)-N′-(3,4-dichlorophenyl)urea, 2,4,4′-trichloro-2′-hydroxydiphenyl ether (triclosan), 4-chloro-3,5-dimethylphenol, 2,2′-methylenebis(6-bromo-4-chlorophenol), 3-methyl-4-(1-methylethyl)phenol, 2-benzyl-4-chlorophenol, 3-(4-chlorophenoxy)-1,2-propanediol, 3-iodo-2-propynylbutyl carbamate, chlorhexidine, 3,4,4′-trichlorocarbanilide (TTC), antibacterial odorants, thymol, thyme oil, eugenol, clove oil, menthol, mint oil, farnesol, phenoxyethanol, glyceryl monocaprate, glyceryl monocaprylate, glyceryl monolaurate (GML), diglyceryl monocaprate (DMC), N-alkylsalicylamides, for example n-octylsalicylamide or n-decylsalicylamide.

### Perspiration-absorbing Substances

Perspiration-absorbing substances include modified starch, for example Dry Flo Plus (from National Starch), silicates, talc and other substances of similar modification which appear suitable for absorption of perspiration. The inventive formulations may comprise the perspiration-absorbing substances in amounts of 0.1 to 30 wt %, preferably 1 to 20 wt % and especially 2 to 8 wt %—based on the total weight of the cosmetic and/or pharmaceutical formulation.

### UV Light Protection Filters

Suitable UV light protection filters in accordance with the invention are organic substances (light protection filters), liquid or crystalline at room temperature, which are capable of absorbing ultraviolet rays and releasing the energy absorbed again in the form of longer-wave radiation, for example heat. UV filters may be oil-soluble or water-soluble. Examples of typical oil-soluble UV-B filters or broad-spectrum UV-KB filters include:
3-benzylidenecamphor or 3-benzylidenenorcamphor (Mexoryl SDS 20) and derivatives thereof, e.g. 3-(4-methylbenzylidene)camphor;
3-(4′-trimethylammonium)benzylidenebornan-2-one methylsulfate (Mexoryl SO)
3,3′-(1,4-phenylenedimethine)bis(7,7-dimethyl-2-oxobicyclo[2.2.1]heptane-1-methanesulfonic acid) and salts (Mexoryl SX)
3-(4′-sulfo)benzylidenebornan-2-one and salts (Mexoryl SL)
polymer of N-{(2 and 4)-[2-oxoborn-3-ylidene)methyl}benzyl]acrylamide (Mexoryl SW)
2-(2H-benzotriazol-2-yl)-4-methyl-6-(2-methyl-3-(1,3,3,3-tetramethyl-1-(trimethylsilyloxy)disiloxanyl)propyl)phenol (Mexoryl XL)
4-aminobenzoic acid derivatives, preferably 2-ethylhexyl 4-(dimethylamino)benzoate, 2-octyl 4-(dimethylamino)benzoate and amyl 4-(dimethylamino)benzoate;
esters of cinnamic acid, preferably 2-ethylhexyl 4-methoxycinnamate, propyl 4-methoxycinnamate, isoamyl 4-methoxycinnamate, 2-ethylhexyl 2-cyano-3,3-phenylcinnamate (octocrylene);
esters of salicylic acid, preferably 2-ethylhexyl salicylate, 4-isopropylbenzyl salicylate, homomenthyl salicylate;
derivatives of benzophenone, preferably 2-hydroxy-4-methoxybenzophenone, 2-hydroxy-4-methoxy-4′-methylbenzophenone, 2,2′-dihydroxy-4-methoxybenzophenone;
esters of benzalmalonic acid, preferably di-2-ethylhexyl 4-methoxybenzalmalonate;
triazine derivatives, for example 2,4,6-trianilino(p-carbo-2′-ethyl-1′-hexyloxy)-1,3,5-triazine and 2,4,6-tris[p-(2-ethylhexyloxycarbonyl)anilino]-1,3,5-triazine (Uvinul T 150) or bis(2-ethylhexyl) 4,4′-[(6-[4-((1,1-dimethylethyl)aminocarbonyl)phenylamino]-1,3,5-triazine-2,4-diyl)dimino]benzoate (Uvasorb® HEB);
2,2-(methylenebis(6-(2H-benzotriazol-2-yl)-4-(1,1,3,3-tetramethylbutyl)phenol) (Tinosorb M);
2,4-bis[4-(2-ethylhexyloxy)-2-hydroxyphenyl]-6-(4-methoxyphenyl)-1,3,5-triazine (Tinosorb S);
propane-1,3-diones, for example 1-(4-tert-butylphenyl)-3-(4′-methoxyphenyl)propane-1,3-dione;
ketotricyclo(5.2.1.0)decane derivatives,
dimethicodiethyl benzalmalonates (Parsol SLX).

Useful water-soluble UV filters include:
2-phenylbenzimidazole-5-sulfonic acid and the alkali metal, alkaline earth metal, ammonium, alkylammonium, alkanolammonium and glucammonium salts thereof;
2,2-(1,4-phenylene)bis(1H-benzimidazole-4,6-disulfonic acid, monosodium salt) (Neo Heliopan AP);
sulfonic acid derivatives of benzophenones, preferably 2-hydroxy-4-methoxybenzophenone-5-sulfonic acid and salts thereof;
sulfonic acid derivatives of 3-benzylidenecamphor, for example 4-(2-oxo-3-bornylidenemethyl)benzenesulfonic acid and 2-methyl-5-(2-oxo-3-bornylidene)sulfonic acid and salts thereof.

Useful typical UV A filters are especially derivatives of benzoylmethane, for example 1-(4′-tert-butylphenyl)-3-(4′-methoxyphenyl)propane-1,3-dione, 4-tert-butyl-4′-methoxydibenzoylmethane (Parsol® 1789), 1-phenyl-3-(4′-isopropylphenyl)propane-1,3-dione, and enamine compounds, and also hexyl 2-[4-(diethylamino)-2-hydroxybenzoyl]benzoate (Uvinul® A plus).

The UVA and UVB filters can of course also be used in mixtures. Particularly favorable combinations consist of the derivatives of benzoylmethane, e.g. 4-tert-butyl-4′-methoxydibenzoylmethane (Parsol® 1789) and 2-ethylhexyl 2-cyano-3,3-phenylcinnamate (octocrylene) in combination with esters of cinnamic acid, preferably 2-ethylhexyl 4-methoxycinnamate and/or propyl 4-methoxycinnamate and/or isoamyl 4-methoxycinnamate.

Combinations of this type are advantageously combined with water-soluble filters, for example 2-phenylbenzimidazole-5-sulfonic acid and the alkali metal, alkaline earth metal, ammonium, alkylammonium, alkanolammonium and glucammonium salts thereof.

### Pigments

In addition to the soluble substances mentioned, insoluble light protection pigments, specifically finely dispersed metal oxides and salts, are also useful for this purpose. Examples of suitable metal oxides are especially zinc oxide and titanium dioxide, and additionally oxides of iron, of zirconium, of silicon, of manganese, of aluminum and of cerium, and mixtures thereof. The salts used may be silicates (talc), barium sulfate or zinc stearate. The oxides and salts are used in the form of the pigments for skincare and skin-protecting emulsions, and also for decorative cosmetics. The particles should have a mean diameter of less than 100 nm, preferably between 5 and 50 nm and especially between 15 and 30 nm. They may have a spherical shape, but it is also possible to use those particles which have an ellipsoidal shape or a shape which deviates in some other way from the spherical configuration. The pigments may also be present in surface-treated form, i.e. hydrophilized or hydrophobized. Typical examples are coated titanium dioxides, for example T 805 titanium dioxide (Degussa) or Eusolex® T, Eusolex® T-2000, Eusolex® T-Aqua, Eusolex® AVO, Eusolex® T-ECO, Eusolex® T-OLEO and Eusolex® T-S (Merck). Typical examples are zinc oxides, for example Zinc Oxide neutral, Zinc Oxide NDM (Symrise) or Z-Cote® (BASF) or SUNZnO-AS and SUNZnO-NAS (Sunjun Chemical Co. Ltd.). Suitable hydrophobic coating agents are in particular silicones and specifically trialkoxyoctylsilanes or simethicones. In sunscreen compositions, preference is given to using micropigments or nanopigments. Preference is given to using micronized zinc oxide.

In addition to the two aforementioned groups of primary light protection substances, it is also possible to use secondary light protection agents of the antioxidant type, which interrupt the photochemical reaction chain which is triggered when UV radiation penetrates into the skin. Typical examples thereof are amino acids (e.g. glycine, histidine, tyrosine, tryptophan) and derivatives thereof, imidazoles (e.g. urocanic acid) and derivatives thereof, peptides such as D,L-carnosine, D-carnosine, L-carnosine and derivatives thereof (e.g. anserine), carotenoids, carotenes (e.g. -carotene, -carotene, lycopene) and derivatives thereof, chlorogenic acid and derivatives thereof, lipoic acid and derivatives thereof (e.g. dihydrolipoic acid), aurothioglucose, propylthiouracil and other thiols (e.g. thioredoxin, glutathione, cysteine, cystine, cystamine and the glycosyl, N-acetyl, methyl, ethyl, propyl, amyl, butyl and lauryl, palmitoyl, oleyl, linoleyl, cholesteryl and glyceryl esters thereof), and salts thereof, dilauryl thiodipropionate, distearyl thiodipropionate, thiodipropionic acid and derivatives thereof (esters, ethers, peptides, lipids, nucleotides, nucleosides and salts), and sulfoximine compounds (e.g. buthionine sulfoximines, homocysteine sulfoximine, buthionine sulfones, penta-, hexa-, heptathionine sulfoximine) in very low tolerated doses (e.g. pmol to mol/kg), also (metal) chelating agents (e.g. a-hydroxy fatty acids, palmitic acid, phytic acid, lactoferrin), α-hydroxy acids (e.g. citric acid, lactic acid, malic acid), humic acid, gallic acid, bile extracts, bilirubin, biliverdin, EDTA, EGTA and derivatives thereof, unsaturated fatty acids and derivatives thereof (e.g. gamma-linolenic acid, linoleic acid, oleic acid), folic acid and derivatives thereof, ubiquinone and ubiquinol and derivatives thereof, vitamin C and derivatives (e.g. ascorbyl palmitate, Mg ascorbyl phosphate, ascorbyl acetate), tocopherols and derivatives (e.g. vitamin E acetate), vitamin A and derivatives (vitamin A palmitate), and coniferyl benzoate of benzoin resin, rutinic acid and derivatives thereof, a-glycosylrutin, ferulic acid, furfurylideneglucitol, carnosine, butylhydroxytoluene, butylhydroxyanisole, nordihydroguaiacic acid, nordihydroguaiaretic acid, trihydroxybutyrophenone, uric acid and derivatives thereof, mannose and derivatives thereof, superoxide dismutase, zinc and derivatives thereof (e.g. ZnO, ZnSO4), selenium and derivatives thereof (e.g. selenomethionine), stilbenes and derivatives thereof (e.g. stilbene oxide, trans-stilbene oxide) and the derivatives (salts, esters, ethers, sugars, nucleotides, nucleosides, peptides and lipids), suitable in accordance with the invention, of these specified active ingredients.

### Non-ionic Emulsifiers

Non-ionic emulsifiers may also be present in combination with the anionic emulsifiers. Examples of these non-ionic emulsifiers are fatty alcohol polyglycol ethers (Eumulgin S 2, Eumulgin S 21, Eumulgin B1, Eumulgin B2, Cremophor A 25, Eumulgin B3, BASF and emulsifier compounds such as Emulgade 1000 NI, Lanette WAX AO, Emulgade SE PF, Emulgade NLB, alkylphenol polyglycol ethers, fatty acid polyglycol esters such as Emulgade 165, Cremophor GS 32, fatty acid amide polyglycol ethers, fatty amine polyglycol ethers, alkoxylated triglycerides, mixed ethers or mixed formals, optionally partially oxidized alk(en)yl oligoglycosides or glucuronic acid derivatives, fatty acid N-alkylglucamides, protein hydrolyzates, especially wheat-based plant products, polyol fatty acid esters, sugar esters (such as Emulgade PL 68/50, Emulgade Sucro from BASF), sorbitan esters, polysorbates and amine oxides. If the nonionic surfactants comprise polyglycol ether chains, these may have a conventional homolog distribution, but preferably have a narrowed homolog distribution. The proportion of the non-ionic emulsifiers is generally about 0.01 to 10.00 wt %, preferably 0.05 to 5.00 wt % and especially 0.05 to 3.00 wt %.

### Thickeners

Suitable thickeners include, for example, aerosil types (hydrophilic silicas), sheet silicates such as magnesium aluminum silicate, polyvinyl alcohol, polyvinylpyrrolidone and bentonites such as Bentone® Gel VS-5PC (Rheox), taurates and derivatives thereof, polyurethanes, polyacrylamides, PVM/MA copolymers and mixtures.

### Biogenic Active Ingredients

Biogenic active ingredients are understood to mean, for example, tocopherol, tocopherol acetate, tocopherol palmitate, ascorbic acid, (deoxy)ribonucleic acid and the fragmentation products thereof, β-glucans, retinol, bisabolol, allantoin, phytantriol, panthenol, AHA acids, amino acids, ceramides, pseudoceramides, essential oils, plant extracts, such as, for example, Prunus extract, bambara nut extract and vitamin complexes.

Examples of useful insect repellents include N,N-diethyl-m-toluamide, 1,2-pentanediol or ethyl 3-(N-n-butyl-N-acetylamino)propionate, which is sold under the Insect Repellent® 3535 name by Merck KGaA, and butylacetylaminopropionates.

Dihydroxyacetone is suitable as self-tanning agent. Possible tyrosine inhibitors, which prevent the formation of melanin and are applied in depigmenting compositions, are, for example, arbutin, ferulic acid, kojic acid, coumaric acid and ascorbic acid (vitamin C).

Examples of suitable preservatives are phenoxyethanol, formaldehyde solution, parabens, pentanediol or sorbic acid, and also the silver complexes known under the name Surfacine®, and the additional substance classes listed in Annex 6, parts A and B, of the Cosmetics Directive.

Perfume oils include mixtures of natural and synthetic odorants. Natural odorants are extracts of flowers, stems and leaves, fruit, fruit shells, roots, wood, herbs and grasses, needles and branches, resins and balsams. Additionally possible are animal raw materials, such as, for example, civet and castoreum, and also synthetic odorant compounds of the ester, ether, aldehyde, ketone, alcohol and hydrocarbon type.

Possible pearlizing waxes, in particular for use in surface-active formulations, are, for example: alkylene glycol esters, especially ethylene glycol distearate; fatty acid alkanolamides, especially coconut fatty acid diethanolamide; partial glycerides, especially stearic acid monoglyceride; esters of polyvalent, optionally hydroxy-substituted, carboxylic acids with fatty alcohols having 6 to 22 carbon atoms, especially long-chain esters of tartaric acid; fatty substances, such as, for example, fatty alcohols, fatty ketones, fatty aldehydes, fatty ethers and fatty carbonates, which have in total at least 24 carbon atoms, especially laurone and distearyl ether; fatty acids, such as stearic acid, hydroxystearic acid or behenic acid, ring-opening products of olefin epoxides having 12 to 22 carbon atoms with fatty alcohols having 12 to 22 carbon atoms and/or polyols having 2 to 15 carbon atoms and 2 to 10 hydroxyl groups, and mixtures thereof.

Use may be made, as superfatting agents, of substances such as, for example, lanolin and lecithin, and also polyethoxylated or acylated lanolin and lecithin derivatives, polyol fatty acid esters, monoglycerides and fatty acid alkanolamides, the latter simultaneously serving as foam stabilizers.

Use may furthermore be made, in order to improve the flow behavior, of hydrotropes, such as, for example, ethanol, isopropyl alcohol or polyols. Polyols which are suitable here preferably have 2 to 15 carbon atoms and at least two hydroxyl groups. The polyols may comprise still other functional groups, in particular amino groups, or be modified with nitrogen.

The invention will now be elucidated hereinafter with reference to the examples.

### EXAMPLES

The textured compositions 1 to 6 shown in the following table 1 are prepared as follows:

The gel former of phase 3 is dispersed in water with stirring and is pre-swelled with addition of phase 4 with stirring until a homogeneous gel forms. The constituents of phase 1 and phase 2 are heated to 75 to 85° C. and combined with stirring. After the phase combination, the homogeneous gel (phase 3 and phase 4) is added with slow stirring. The combined phases are slowly stirred at a speed of <600 rpm until room temperature is reached. The stirring speed during stirring is preferably 300 rpm. At approximately 35° C. the constituents of phase 5 are added.

TABLE 1

| Phase | Commercial product | Chemical designation | 1 | 2 | 3 | 4 | 5 | 6 |
|---|---|---|---|---|---|---|---|---|
| 1 | Emulgade Sucro Plus | Sucrose polystearate, cetyl | | | | 1.00 | 1.50 | 2.00 |
| | | palmitate | | | | | | |
| 1 | Eumulgin SG | Sodium stearoyl glutamate | 0.01 | 0.05 | 0.05 | | | |
| 1 | Cutina FS 45 | Stearic acid, palmitic acid | 3.00 | | | | | |
| 1 | Lanette O | Cetearyl alcohol | | 3.00 | | 4.00 | 3.00 | 3.00 |
| 1 | Lanette 22 | Behenyl alcohol | | | 3.00 | | | |
| 1 | Cetiol LC | Coco caprylate/caprate | | | 4.50 | 7.50 | 5.00 | 5.00 |
| 1 | Cetiol C5 | Coco caprylate | 4.00 | 2.50 | 3.00 | | 3.00 | 2.00 |
| 1 | Cetiol CC | Dicaprylyl carbonate | | 1.50 | | | | |
| 1 | Cetiol SB 45 | Butyrospermum Parkii | | | | | | 0.50 |
| | | (Shea Butter) | | | | | | |
| 1 | Cetiol Sensoft | Propylheptyl caprylate | 2.00 | 1.50 | | 2.50 | | |
| 1 | Cetiol Ultimate | Undecane (and) tridecane | | | | 2.00 | | |
| 1 | Cetiol 4all | Dipropylheptyl carbonate | 2.00 | | | | 2.50 | 2.00 |
| 1 | Myritol 312 | Caprylic/capric triglyceride | | 2.00 | | | | |
| 2 | Demin. water | Water | 50.26 | 50.95 | 51.27 | 47.70 | 47.00 | 47.50 |
| 2 | Glycerol | Glycerol | 3.00 | 3.00 | 3.00 | 3.00 | 3.00 | 3.00 |
| 3 | Rheocare C Plus | Carbomer | 0.20 | 0.20 | 0.20 | 0.20 | 0.20 | 0.20 |
| 3 | Demin. water | Water | 33.13 | 33.00 | 33.13 | 30.00 | 29.70 | 33.00 |
| 4 | KOH 20% | Potassium hydroxide | 0.40 | 0.30 | 0.35 | 0.30 | 0.30 | 0.30 |
| 5 | Parfum “Always | Always the sun fragrance | 0.50 | 0.50 | 0.50 | 0.50 | 0.50 | 0.50 |
| | the sun” | from Symrise | | | | | | |
| 5 | Patch2O | Water, glycerol, glyceryl | | | | | 3.00 | |
| | | polyacrylate, trehalose, | | | | | | |
| | | urea, serine, pentylene | | | | | | |
| | | glycol, algin, caprylyl | | | | | | |
| | | glycol, sodium hyaluronate, | | | | | | |
| | | pullulan, disodium phosphate, | | | | | | |
| | | potassium phosphate | | | | | | |
| 5 | Euxyl K 320 | 2-Phenoxyethanol, | | | 1.00 | 1.30 | 1.30 | 1.00 |
| | | methyl-4-hydroxybenzoate, | | | | | | |
| | | ethyl-4-hydroxybenzoate | | | | | | |
| 5 | Protectol PE | Phenoxyethanol | 1.00 | 1.00 | | | | |
| 5 | Sensiva SC 50 | Ethylhexylglycerin | 0.50 | 0.50 | | | | |
| | Viscosity | Brookfield RVF, Spindle | 112500 | 100000 | 75000 | 150000 | 112500 | 112500 |
| | | TE, 4 rpm with Helipath | | | | | | |
| | pH | | 6.5 | 6.4 | 6.3 | 6.4 | 6.4 | 6.7 |

The transmittance values of the compositions 1 to 6 were measured as follows:

The transmittance values of the compositions 1 to 6 were measured with a Lumifuge (LUM GmbH, Berlin) at the lowest possible rotational speed of 200 rpm. Once the instruments had been standardized, cuvettes (type PA 110-13405) with an optical path of 2 mm were filled with the sample and the percentage of transmitted light was averaged over a height of 10 mm.

The transmittance values determined for the compositions are found in table 2.

TABLE 2

| | Transmission at 25° C. | Std. |
|---|---|---|
| O/W emulsion - commercial | 3.77 | 0.01 |
| product soft creme | | |
| Textured composition 6 | 5.69 | 0.01 |
| Textured composition 5 | 6.09 | 0.01 |
| Textured composition 4 | 6.12 | 0.01 |
| Textured composition 3 | 7.28 | 0.01 |
| Textured composition 2 | 9 | 0.02 |
| Textured composition 1 | 10.18 | 0.02 |
| Demin. water | 91.19 | 0.14 |

In comparison to a commercial product, soft creme, the inventive textured compositions have higher transmittance values. This means that the inventive textured compositions are to some extent significantly more transparent than the commercial product.

1. A textured composition which forms a lamellar and/or crystalline gel network in an oil-in-water system and which comprises:
an emulsifier combination comprising
0.01 to 3.00 wt % of at least one anionic emulsifier, and
0.15 to 9.00 wt % of at least one consistency regulator,
0.01 to 5.00 wt % of at least one hydrocolloid,
3.00 to 15.0 wt % of at least one oil and/or wax, and
made up to 100 wt % with water,
wherein the anionic emulsifier comprises at least one emulsifier selected from the group consisting of salts of fatty acids, salts of stearoyl lactic acid, salts of stearoyl glutamic acid or alkyl glutamates, alkyl phosphates, alkyl sulfates, alkyl sarcosinates, salts of alkylsulfosuccinic acid, and salts of citric acid esters, a ratio of anionic emulsifier to consistency regulator in a range from 10:333 to 10:6, and the oil or the mixtures thereof has a medium polarity with an interfacial tension IFT of 25 to 45 mN/m.

2. The composition according to claim 1, wherein the at least one consistency regulator is present at a concentration of 0.5 to 6.00 wt % and is selected from the group consisting of of fatty alcohols, fatty acids (unsaponified), glyceryl mono-, di- and/or tri-fatty acid esters, sorbitan mono-, di-, tri- and/or poly-fatty acid esters, sucrose mono-, di-, tri- and/or poly-fatty acid esters, pentaerythrityl mono-, and/or di-fatty acid esters.

3. The composition according to claim 1, wherein the at least one hydrocolloid is present at a concentration of 0.1 to 1.00 wt %.

4. The composition according to claim 1, wherein the at least one oil is present at a concentration of 5.00 to 15.00 wt %.

5. The composition according to claim 1, wherein the fatty alcohol comprises at least one fatty alcohol of the formula (I)
R—OH (I),
where R signifies an aliphatic, linear or branched alkyl radical having 6 to 22 carbon atoms, having 0 and/or 1, 2 or 3 double bonds.

6. The composition according to claim 1, wherein the hydrocolloid is selected from the group consisting of acrylic acid and acrylic acid derivatives, carbohydrates such as cellulose, and natural rubber and derivatives thereof, taurates and derivatives thereof, polyurethanes, polyacrylamides, PVM/MA copolymers, VP copolymer, VA copolymers, and mixtures thereof.

7. The composition according to claim 1, wherein the oil or wax is selected from the group consisting of fatty acid esters, hydrocarbons, Guerbet alcohols, tri- or partial glycerides, mono-/dialkyl ethers, mono-/dialkyl carbonates, oil-soluble UV filters, fatty alcohol ethers, microcrystalline waxes, mineral oil, silicone oil, natural vegetable oils, and mixtures thereof.

8. The composition according to claim 1, wherein the emulsifier combination of anionic emulsifier and consistency regulator is 1.5 to 6.0 wt %.

9. The composition according to claim 1, wherein the textured composition has a translucency with a transmittance of 3.9 to 50% at 25° C.

10. A textured composition which forms a finely dispersed droplet distribution having lamellar and/or crystalline structures in an oil-in-water system and which comprises:
an emulsifier combination comprising
0.01 to 5.00 wt % of at least one anionic emulsifier,
0.15 to 9.00 wt % of at least one consistency regulator,
0.01 to 5.00 wt % of at least one hydrocolloid,
3.00 to 15.0 wt % of at least one oil and/or wax, and
made up to 100 wt % with water,
wherein the anionic emulsifier comprises at least one emulsifier selected from the group consisting of salts of fatty acids, salts of stearoyl lactic acid, salts of stearoyl glutamic acid or alkyl glutamates, alkyl phosphates, alkyl sulfates, alkyl sarcosinates, salts of alkylsulfosuccinic acid, and salts of citric acid esters, and the emulsifier combination is 2.00 to 8.00 wt %, and a ratio of anionic emulsifier to consistency regulator is 1:1 to 1:2, and the oil or the mixtures thereof has a medium polarity with an interfacial tension IFT of 25 to 45 mN/m.

11. A process for preparing a textured composition according to claim 1, wherein the constituents are combined after heating to 75 to 85° C., are slowly stirred at a speed of less than 600 rpm, until a temperature of 55 to 75° C. is reached, the mixture is subsequently homogenized, and then is stirred at a speed of less than 600 rpm, until room temperature is reached.

12. The textured cosmetic composition according to claim 1,
further comprising cosmetically active constituents from the group consisting of pigments, plant extracts, peptides, proteins, marine atelocollagen, phytoceramides, phytosterols, polyphenols, polyols, urea, hyaluronic acid, sugars and sugar derivatives, sodium PCA, vitamins, UV light protection filters, antioxidants, biogenic active ingredients, self-tanning agents, preservatives, complexing agents, perfume oils, vegetable oils, antiperspirants, esterase inhibitors, neutralizing agents, bactericides, and mixtures thereof.

13. The textured cosmetic composition according to claim 12, wherein said composition is in the form of creams, milk, lotions, gels, sticks, conditioners, sprays, serum, aerosol foam, pump foam, pastes, or waxes.

14. A textured cosmetic composition according to claim 1 for use in skincare for the face, body, hands, area around the eyes and décolletage, as sunscreen compositions and for skincare for babies, and for the care of unwell and sensitive skin.

15. The composition according to claim 5 wherein R has 14 to 20 carbon atoms.

16. The process according to claim 11 wherein stirring is performed at a speed of less than/equal to 300 rpm.
